(19) United States
(12) Patent Application Publication
(10) Pub. No.: US 2026/0288123 A1
(43) Pub. Date: Sep. 24, 2026

(54) WATER PURIFICATION PLANT FOR THE NEEDS-OPTIMISED EXECUTION OF OPERATIONS

(71) Applicant: FRESENIUS MEDICAL CARE DEUTSCHLAND GMBH, Bad Homburg (DE)

(72) Inventors: Yahya FAROON, Bad Homburg (DE); Andreas HEMM, Bad Homburg (DE); Frank STRYEWSKI, Bad Homburg (DE); Sascha SCHMITTEL, Bad Homburg (DE); Mirko MAURER, Bad Homburg (DE)

(73) Assignee: FRESENIUS MEDICAL CARE DEUTSCHLAND GMBH, Bad Homburg (DE)

(21) Appl. No.: 19/480,035

(22) PCT Filed: Apr. 25, 2024
(86) PCT No.: PCT/EP2024/061385

§ 371 Date: Oct. 30, 2025

(30) Foreign Application Priority Data
May 4, 2023 (DE) 10 2023 111 567.7

(51) Int. Cl. G05B 19/418; C02F 1/44

(52) U.S. Cl. CPC G05B 19/41865; C02F 1/441; C02F 2209/001; C02F 2209/44

(57) ABSTRACT

The present invention relates to a water treatment system for the production of medical solution, in particular dialysis water, which can be operated in a first operating mode and a second operating mode, comprising a control unit, characterized in that the control unit is programmed to detect, preferably on the basis of sensor data, whether operation of the water treatment system and/or at least one component thereof in the second operating mode is necessary and, if operation of the water treatment system in the second operating mode has been detected to be necessary, to determine whether switching from the first operating mode to the second operating mode is possible at a current point in time, and if the determination is that switching to the second operating mode is possible at the current time, to issue a control instruction to the water treatment system and/or at least one component thereof to operate it in the second operating mode, and if the determination is that switching to the second operating mode is not possible at the current time, to determine a future time at which the control unit issues a control instruction to the water treatment system and/or at least one component thereof to operate it in the second operating mode.

The present invention relates to a water treatment system for the demand-optimized execution of operating sequences, in particular for the coordinated integration of maintenance modes into a treatment operation of the water treatment system.

Procedures for kidney replacement treatment, e.g. peritoneal dialysis or hemodialysis treatments, usually require special dialysate solutions which are produced or mixed from high-purity water. In dialysis centers, this water is usually provided at great expense via a reverse osmosis (RO) system, sometimes with several upstream and downstream filter stages. Once the high-purity water has been produced by the water treatment system, it is enriched with glucose and various ions, for example, and/or mixed with prefabricated dialysis concentrates. The aim here is to ensure that different substance concentrations prevail between the blood and dialysate sides in a dialysis treatment in order to achieve a substance exchange and water reduction as part of the treatment process.

The coordination of the individual operational and technical processes is crucial here. Cleaning, regeneration or mixing processes within a water treatment system always require a temporal alignment with upcoming treatment processes in the dialysis center or maintenance processes of the water treatment system itself. Coordination of different system components and processes is essential here to ensure that life-saving dialysis treatments run smoothly.

At present, typical routine procedures or maintenance work on the water treatment system, e.g. necessary filter backwashing, are scheduled to ensure that dialysis runs as smoothly as possible. As a rule, dates are chosen to be late at night or early in the morning, although a more efficient choice of date, e.g. during a treatment break during the day, would be possible.

Furthermore, in many cases such routine procedures are not always absolutely necessary and could be delayed, e.g. if the capacity of a filter is not yet exhausted. Traditionally, however, a large number of routine measures are carried out on suspicion and unnecessarily, resulting in increased water and energy consumption.

There is therefore a need for technical means for time-, water-and energy-efficient control of water treatment systems, for example in the context of planned dialysis treatments in a dialysis center.

Against this background, the present invention has the object of mitigating or even completely eliminating the disadvantages of the prior art.

This object is achieved by a water treatment system having the features of claim 1. Advantageous further embodiments of the invention are the subject-matter of the subclaims.

Accordingly, a water treatment system for producing medical solution, in particular dialysis water, is provided, which can be operated in a first operating mode, for example a treatment mode or a mode for providing medical solution for dialysis treatment, and a second operating mode, for example a maintenance mode for filter regeneration.

The water treatment system comprises a control unit programmed to detect, preferably on the basis of sensor data, whether operation of the water treatment system and/or at least one component thereof is required in the second operating mode.

In other words, for example, a filter is not regenerated on suspicion, but only if the control unit detects, for example on the basis of sensor data reflecting a remaining filter capacity, that filter regeneration is actually required.

If operation of the water treatment system in the second operating mode has been detected to be necessary, for example because a filter capacity is only 10%, the control unit determines whether it is possible to switch from the first operating mode to the second operating mode at a current point in time. In other words, it first checks whether the filter can be regenerated immediately, for example because the water treatment system is not currently being operated to produce medical solution.

If the determination shows that switching to the second operating mode is possible at the current time, the control unit preferably issues a control instruction to the water treatment system and/or at least one component, e.g. the filter module to be regenerated, to operate it in the second operating mode (e.g. filter regeneration).

However, if the determination shows that switching to the second operating mode is not possible at the current time, for example because an ongoing dialysis treatment has been detected for which medical solution must be generated, the control unit determines a future time at which the control unit issues a control instruction to the water treatment system and/or at least one component thereof to operate it in the second operating mode.

For example, the control unit identifies a time window after the end of the current treatment and controls the filter module for filter regeneration in this time window.

Thus, the control unit is preferably configured to automatically and without human interaction determine a future time window or a point in time in which the water treatment system and/or at least one component is operated in the second operating mode. Maintenance processes can thus be flexibly and automatically integrated into a planned daily routine.

In addition, by determining whether, for example, a maintenance process is absolutely necessary, unnecessary maintenance processes can be avoided, for example at fixed intervals (the filter is regenerated every 2 weeks, regardless of its residual capacity), thereby conserving resources.

A first operating mode here preferably refers to an operating mode which prevents the execution of maintenance processes of the water treatment system or components thereof, for example an operation for the generation of dialysis water and/or medical solution, in particular dialysate, for an ongoing dialysis treatment.

A second operating mode here preferably refers to an operating mode for carrying out a maintenance process of the water treatment system or at least one component thereof and/or an internal system process, such as a mixing process of a dialysis concentrate with generated dialysis water. A second operating mode can, for example, relate to a backwashing or regeneration process of a filter, a refilling of concentrate, a replacement process of a component, etc.

An “internal system process” is preferably a process that relates to the internal operation of the water treatment system, for example a mixing process in which liquid or solid dialysis concentrate is mixed with dialysis water to produce dialysate. The finished dialysate is then dispensed to at least one treatment device in the first operating mode.

The control unit may further be programmed to compare sensor data relating to at least one system-specific and/or component-specific parameter with at least one threshold value stored in a database when determining whether operation of the water treatment system and/or at least one component thereof is required in the second operating mode.

For example, a pressure in a line upstream and/or downstream of a filter can be detected. If the pressure in the line upstream of the filter exceeds an associated threshold value, it can be concluded that a backwash process is required. If the pressure in the line downstream of the filter falls below an associated threshold value, it can be concluded that a backwash process is required. Furthermore, a pressure drop across the filter at a defined minimum flow can be detected.

In this regard, detection as to whether a certain process is required (e.g. pressure above 1 bar: yes; pressure below 1 bar: no) can either be effected digitally, or a more differentiated evaluation can be carried out, in which a necessity value is calculated, which reflects, for example, on a scale of 0 % to 100 %, how necessary it is to carry out a certain process, or the second operating mode. For example, a pressure above 2 bar measured upstream of the filter may correspond to a 75% necessity value (filter clogged); a pressure between 0.5 and 1 bar may correspond to a 50% necessity value (filter somewhat clogged but still acceptable); and a pressure below 0.5 bar may correspond to an 80% necessity value (filter damaged). If the pressure is measured downstream of the filter, reduced measured pressure values may indicate clogging of the filter. If a pressure drop across the filter is detected at a defined minimum flow, a relatively large pressure drop across the filter may indicate that the filter is clogged.

The control unit may further be programmed to determine, when determining whether operation of the water treatment system and/or at least one component thereof in the second operating mode is required, preferably taking into account sensor data relating to at least one system-specific and/or component-specific parameter and/or at least one threshold value for the second operating mode stored in a database, an urgency value reflecting an urgency with which the water treatment system and/or at least one component thereof is to be operated in the second operating mode.

For example, after it has been detected that a corresponding operation with a certain necessity value (e.g. 80%) is required in a second operating mode, an associated urgency value can be determined for this second operating mode, for example on the basis of historical data.

For example, the historical data may indicate that an urgency value of 90% (e.g. on a scale of 0% to 100%) is appropriate for a filter backwash process with a necessity value of 75%, as experience has shown that the water treatment system will fail promptly if the filter is not backwashed promptly in this situation.

The second operating mode can be linked in the control unit with the associated necessity value and the associated urgency value.

The determined urgency values also allow a specific second operating mode relating to a component (e.g. filter backwashing) to be prioritized over another second operating mode relating to another component (e.g. replacement of a membrane in a reverse osmosis system), which cannot be carried out in parallel.

The control unit can be programmed to determine an urgency value for each of the components and/or for each of the second operating modes in a case in which it has been detected that it is necessary to operate a plurality of components of the water treatment system in a second operating mode in each case, preferably taking into account sensor data with regard to at least one component-specific parameter and/or at least one threshold value stored in a database.

For example, filter backwashing is linked to an urgency value of 80% and the replacement of a membrane in a reverse osmosis system is linked to an urgency value of 50%. In this case, the filter backwash is prioritized and the control unit issues a control instruction to backwash the filter at the next possible time identified.

In other words, the control unit may further be programmed, when it has been detected that multiple components should be operated in a second operating mode, to select the component and/or the second operating mode associated with the most urgent or highest urgency value and issue a control instruction at the determined future time to operate the selected component in the selected second operating mode.

When automatically determining the future time for issuing the control instruction, planning data is also preferably taken into account, which reflects a planned operation of the water treatment system and includes, for example, planned treatments.

The control unit can therefore be programmed to take into account planning data reflecting a planned future operation of the water treatment system when determining the future time for issuing the control instruction, wherein the planning data includes at least one future time period in which the water treatment system is operated in the first operating mode and/or a further operating mode which prevents switching the operation of the water treatment system to the second operating mode.

Certain planned processes could therefore block or disable switching the operation of the water treatment system or at least one component thereof to the second operating mode.

The other operating mode can, for example, be a mixing process that must be carried out before a planned treatment. Switching the operation of the water treatment system to a second operating mode for filter backwashing, for example, is therefore not possible either during treatment (e.g. first operating mode) or during the mixing process (e.g. further operating mode) and the time windows of these operating modes are therefore blocked for the second operating mode for filter backwashing.

Alternatively or additionally, the planning data for the operation in the second operating mode can specify a certain temporal relationship to an operation in the first operating mode, preferably that the second operating mode is to be carried out before or after a time period in which an operation in the first operating mode takes place.

If, for example, a mixing process of concentrate and dialysis water is assumed as the second operating mode, the planning data can specify that this second operating mode must always take place before a planned treatment (e.g. first operating mode).

In the event of conflicting objectives, e.g. where several maintenance processes are to be planned or carried out within the same time window, prioritization can take place. For example, an activated carbon filter can only be backwashed if no concentrate is being mixed. The concentrate mixing can only start once the backwashing of the filter has been completed (and may include a predetermined waiting time). Both processes may only be started if the treatment devices are not in hot disinfection mode (HSD).

If, for example, it is determined that the residual capacity of a filter before backwashing is not sufficient for a mixing process within the concentrate mixing system, the system automatically schedules the filter to be backwashed first and the mixing process to be carried out afterwards, e.g. during a treatment break.

To prevent additional conflicts of objectives, certain components can also include certain buffers. For example, the capacity limit of the activated carbon filter can be set so that backwashing can be delayed for a few hours in an emergency, e.g. if a mixing process is required immediately. In other words, the threshold values can include tolerance ranges when determining the necessity values.

Preferably, there is an automatic feedback between the specific future time of the control instruction and the planning data in such a way that a specific time is automatically integrated into the planning data.

The control unit can thus be programmed to integrate a specific future point in time at which the control unit issues a control instruction to the water treatment system and/or at least one component thereof in order to operate it in the second operating mode into the planning data and thereby automatically generate updated planning data.

Based on the sensor data or the planning data, new blocked time windows can in turn be generated, which can then be included in a future determination of a time for issuing a control instruction and in a generation of control data on which the control instruction is based.

This is the case, for example, if the control unit detects that one or more filters have reached their capacity limit or that the capacity is only below a limit value.

A time window is required for backwashing, which must be allocated by the control unit as soon as possible, for example. In addition to the identified time window, data can then be generated for other components of the water treatment system that block the operation of these components in a second operating mode in this time window. For example, neither the concentrate mixing system nor a treatment device should be activated during a filter backwash.

In other words, the control unit may further be programmed to determine, based on the determined future time at which the control unit issues a control instruction to the water treatment system and/or at least one component thereof to operate in the second operating mode, at least one future time period in which another component of the water treatment system may not be operated in the second operating mode.

In other words, depending on the scheduling of a future time for the execution of a routine operation, the planning data can be dynamically adapted so that, for example, new blocked time windows (also referred to as blocking time or blocking window) are generated.

For example, it is determined that an activated carbon filter needs to be backwashed within the next 24 hours. A date can then be booked for this in the following night, taking into account other blocking times, such as mixing or emptying processes of the concentrate mixing system. At the same time, new blocking times are generated for the planned backwash process for other components of the system, e.g. for the mixing system.

The scheduling or automatic determination of the time or time window for executing a routine process is therefore preferably always selected so that the time window of the routine process does not lie within a time blocking window of another routine process, whereby the respective time blocking windows of the components are covered by planning data.

Certain routine processes cannot take place in parallel for technical reasons, e.g. filter backwashing cannot take place in parallel with ongoing treatment or mixing processes.

For this reason, planning data can include blocking times for various routine and also non-routine processes, on the basis of which the water treatment system can be controlled, for example.

Non-routine processes can, for example, include unscheduled maintenance processes, which may require at least partial shutdown of the water treatment system. Consequently, during shutdown periods, certain routine operations, such as filter backwashing or hot disinfection, are not possible.

Preferably, in a water treatment system according to the invention, the first operating mode corresponds to an operation of the system for generating medical water for an ongoing treatment, in particular a dialysis treatment, and the second operating mode corresponds to a maintenance mode or maintenance operation of at least one component of the water treatment system, preferably a cleaning operation, a regeneration operation or a replacement operation and/or an internal operation of the system for generating medical solution, preferably an operation for mixing dialysis concentrate with water.

The control unit can be configured to communicate wirelessly and preferably via WLAN or Bluetooth with the water treatment system and/or at least one sensor, which provides sensor data relating to the operation of the water treatment system and/or at least one component of the water treatment system. In principle, wired communication between the control unit and the water treatment system and/or at least one sensor is also conceivable.

The control unit can be configured separately from the water treatment system and be arranged in a smartphone, tablet or external computer, for example.

Furthermore, the aforementioned data processing steps can be carried out either by the control unit or by a data processing unit, which can be configured separately from the water treatment system or installed in it. The data processing unit may be part of or separate from the control unit.

Another aspect of the present invention relates to a method for operating a water treatment system for producing medical solution, in particular dialysis water, preferably a water treatment system according to the present invention, which can be operated in a first operating mode and a second operating mode, comprising the steps of:
detecting, preferably on the basis of sensor data, whether operation of the water treatment system and/or at least one component thereof is required in the second operating mode, and
if operation of the water treatment system in the second operating mode has been detected to be necessary, determining whether switching from the first operating mode to the second operating mode is possible at a current time, and
if the determination is that switching to the second operating mode is possible at the current time, issuing a control instruction to the water treatment system and/or at least one component thereof to operate it in the second operating mode, and
if the determination shows that switching to the second operating mode is not possible at the current time, determining a future time at which the control unit issues a control instruction to the water treatment system and/or at least one component thereof to operate it in the second operating mode.

A method according to the invention can comprise one or more of the functionalities disclosed above in the context of a water treatment system and in particular of a correspondingly configured control unit of such a water treatment system as method steps.

All the features disclosed above in the context of a water treatment system according to the invention are thus also applicable to a method according to the invention, even if an explicit disclosure is omitted in order to avoid redundancies.

Another aspect of the present invention relates to a computer program product which contains machine-readable instructions which, when read by a control unit, for example a water treatment system, provide the latter with the functionality of a water treatment system according to the present invention.

Furthermore, the invention relates to a data carrier which contains machine-readable instructions which, when read by a control unit, a processor or a computer, enable the latter(s) to carry out a method according to the invention.

A computer program product according to the invention and/or a data carrier according to the invention can be used, for example, for retrofitting a conventional water treatment system according to the present invention.

In other words, aspects of the present invention can be described as follows:

One aspect of the present invention relates to a water treatment system for producing medical water, in particular dialysis water, comprising:
at least one module for treating water, preferably a reverse osmosis system,
at least one sensor for detecting at least one parameter of water quality and/or operation of the water treatment system,
at least one data processing unit which is programmed to receive the at least one parameter from the at least one sensor as well as planning data with regard to a planned operation of the water treatment system and to generate at least one control instruction on the basis of the received data, and
a control unit which is programmed to receive the at least one control instruction from the data processing unit and to control at least one component of the water treatment system in accordance with the control instruction.

The technical problem at hand is thus preferably solved by an efficient water treatment system (also referred to as a water treatment system) for demand-optimized planning of routine processes. The water treatment system can comprise various components, e.g. a reverse osmosis (RO) system, filter stages, valves, water softeners and/or a concentrate mixing system, each of which is connected to one another via one or more fluid lines, at least one sensor for detecting one or more operating or water quality parameters, and a control unit.

The at least one sensor preferably transmits the measured values or sensor data generated therefrom to a data processing device, wherein the data processing device may also be included in the water treatment system or the control unit.

The data processing device also receives planning data, wherein the planning data can include requirement data and/or blocking data.

Taking into account the planning data and sensor data, the data processing device preferably generates a control instruction, wherein the control unit is configured to receive and read the control instruction and based thereon, to control at least one component of the water treatment system based on the control instruction.

Processes corresponding to a second operating mode can be routine processes such as maintenance or servicing processes of individual components of the water treatment system, such as backwashing processes of one or more filter stages, mixing processes within a concentrate mixing system or hot disinfection of the piping systems (HSD). Filter backwashing is particularly essential for activated carbon filters, as these become clogged with particles after a certain period of use and consequently offer insufficient filter performance and fluid flow. In such a case, the capacity of the activated carbon filter is exhausted.

By means of appropriate sensor technology, e.g. by means of flow sensors, which may be located in the filter heads, for example, the residual capacity (e.g. in the form of a residual operating time up to the capacity limit) of an activated carbon filter can be determined by the data processing device, preferably using historical data, and stored, for example, in a request file in a database, preferably as threshold value. The threshold value can be used to determine whether maintenance of the filter is required (e.g. from less than 10% filter capacity).

Depending on the filter type, however, the pressure drop across the filter is not a suitable parameter.

In principle, it is therefore conceivable that the water quality can be a criterion with regard to certain marker substances, which in turn are recorded by corresponding sensors. In other words, it is possible to detect whether the filter is still reliably removing certain substances from the water. According to one embodiment, the at least one data processing unit is part of the water treatment system or separate from it.

The data processing device can also be configured as part of the water treatment system. Alternatively, the data processing device is separate from the water treatment system and communicates with it preferably via wireless/wired communication means (Ethernet, Wi-Fi, 5G, The sensors can also be connected to the data processing device via such wireless communication means.

It is also conceivable that the sensors are connected to each other via a mesh network and only communicate with the nearest devices within the mesh network.

One of the advantages of this is that larger distances, e.g. between the data processing device and the most distant sensor in the mesh network, can be bridged wirelessly.

The data processing device can be, for example, a computer, a server or another type of computing unit (e.g. FPGA, RasPI, Arduino, etc.), wherein the computing unit is configured to receive, read in and process planning data and sensor data.

A control instruction can be generated by the data processing unit and/or control unit on the basis of different criteria by comparing the planning data and the sensor data.

Criteria can include, for example, efficiency criteria or availability criteria.

For example, backwashing processes of different filter stages can be coordinated in terms of their residual capacity (and therefore necessity and/or urgency) so that the water treatment system is controlled in a demand-oriented and energy-and water-saving manner.

Since filter backwashing is only required when the filter capacity is exhausted or several filters are backwashed at the same time, efficient control of the water treatment system is possible.

On the other hand, from the point of view of availability criteria, certain routine processes such as backwashing, for example, can be brought forward although the filter capacity limit has not been undercut.

From the point of view of availability, the water treatment system is thus always able to react to changing conditions, e.g. longer or changed treatment times, and provide high-purity water.

The planning data taken into account can include requirement data and/or blocking data.

Requirement data preferably includes predetermined specifications, which can include the type and scope as well as the necessity of certain routine processes.

For example, the request data can specify that a mixing process in the concentrate mixing system should always take place before a treatment is entered. The start or end of a routine process, e.g. a mixing process, can be defined in advance in relative terms, i.e. a certain time before the next routine process starts, or in absolute terms, i.e. always at a certain time of day.

On the other hand, requirement data can include, for example, minimum filter capacities or limit values for certain marker substances in the water, so that a routine process, e.g. a filter backwash, is automatically planned or executed as soon as one or more of the stored criteria are met and it has been detected that the process is necessary.

Requirement data can also include further specifications, e.g. with regard to a backwash or mixing time or a maximum operating time for a component of the water treatment system.

The at least one sensor of the water treatment system that generates sensor data may measure at least one of a temperature, a conductivity, an optical transmission of the water treated by the water treatment system and/or a pressure in a pipe, a performance of a component of the water treatment system, a time period since a last maintenance of at least one component of the water treatment system, or another parameter reflecting the operation of the water treatment system and/or the performance of at least one component thereof.

Preferably, a control instruction issued at the specific future point in time relates to the execution of a routine operation.

Preferably, the planning data relates at least in part to the execution of a medical treatment to be performed, preferably a dialysis treatment.

Preferably, the control instruction contains a future time window in which the control unit has to control at least one component of the water treatment system in accordance with the control instruction. In other words, the control instruction cannot be issued only when the specified future time window is reached, but can be issued in advance and only executed when the specified future time window is reached.

At this point, it should be pointed out that the present invention is not limited to the feature combinations explicitly mentioned herein, but that the features disclosed in the present application may also be claimed in any other combinations or in isolation.

Further advantages, features and effects of the present invention are apparent from the following description of embodiments of the invention with reference to the figures, in which identical reference signs denote identical or similar components.

The figures show in:

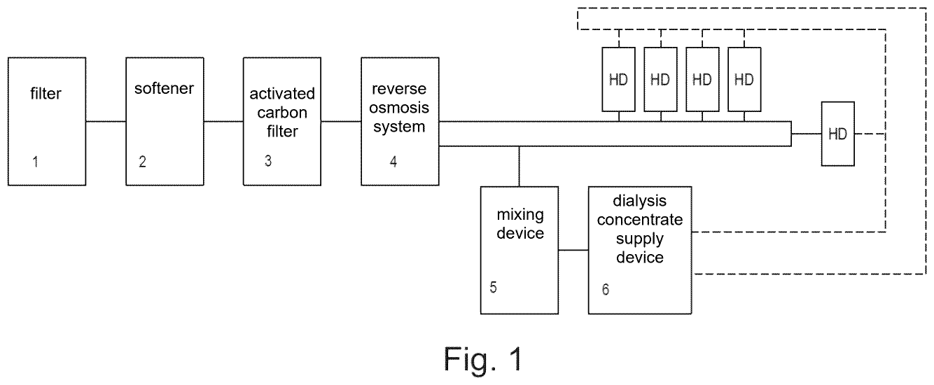

FIG. 1 a water treatment system according to the invention, which can be used, for example, in a dialysis center, and

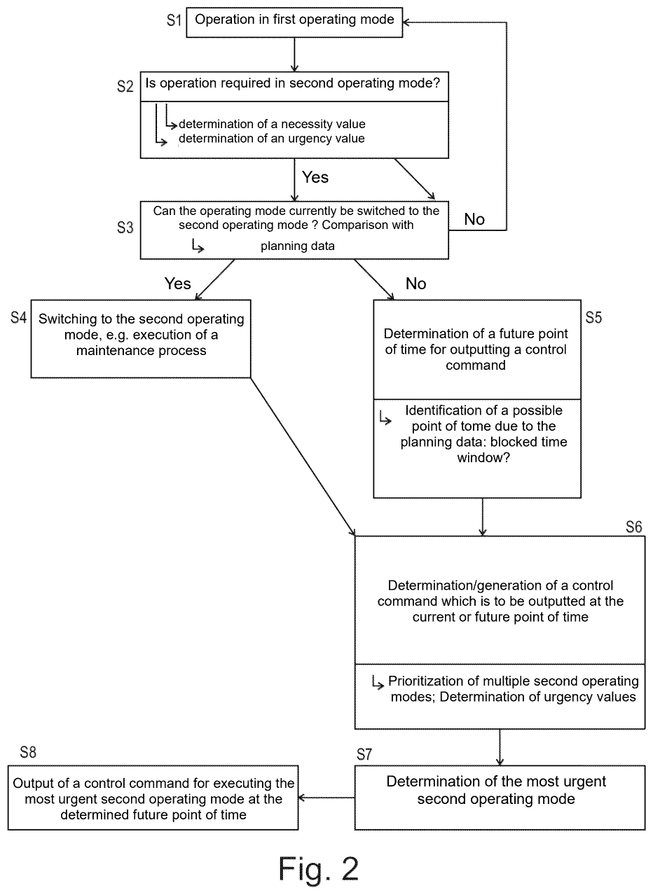

FIG. 2 a process flow which is carried out by a control unit of a water treatment system according to the invention, for example.

As shown in FIG. 1, a water treatment system of one embodiment comprises a filter 1 and/or a pre-filtration stage, a softener 2, an activated carbon filter 3 and a reverse osmosis system 4 (RO), by means of which water, for example tap water, is treated to produce ultrapure water/dialysis water.

The dialysis water output by the RO system 4 is mixed with a liquid or solid concentrate in a mixing device 5 to provide dialysis concentrate, which is delivered to several (haemo)dialysis machines (HD) by means of a dialysis concentrate delivery device 6 and a ring line. The dialysis machines produce dialysate from the dialysis concentrate. Alternatively, it would also be conceivable to supply dialysate to the dialysis machines instead of dialysis concentrate.

The operation of the water treatment system should therefore be coordinated with the treatments carried out by the dialysis machines.

FIG. 2 shows a process flow which is carried out by a control unit of a water treatment system according to the invention, for example.

First, in step S1, the water treatment system is operated in a first operating mode, for example to provide dialysis concentrate for an ongoing treatment.

In step S2, the control unit checks whether operation in a second operating mode, for example a maintenance mode (e.g. filter backwash), is required.

For this purpose, the control unit can determine a necessity value and/or an urgency value for the second operating mode.

In the example shown in FIG. 2, the second operating mode is, for example, a filter backwash.

For example, a pressure increased to 2 bar upstream of the filter and detected by a sensor results in a necessity value of 70% and a comparison with historical data of the water treatment system results in an urgency value of 90%, which indicates that the filter backwash should be carried out as soon as possible so as not to jeopardize the operation of the water treatment system.

Step S3 then checks whether the filter backwash can be carried out directly at the current time. This can be based on sensor data (“Is the system currently pumping dialysis concentrate into the ring main?” If yes, then ongoing treatment) or on planning data that is stored in a database, for example (“treatment planned from 2 pm to 5 pm”; current time is 4 pm, i.e. ongoing treatment). If it turns out that it is possible to switch directly to a second operating mode, then in step S4, for example, the filter backwash is preferably carried out directly and a specific current time window is provided for this second operating mode.

If a current, direct switchover to the second operating mode is not possible, e.g. due to an ongoing treatment, a future time or a time window for carrying out the filter backwash is determined in step S5.

When determining the future time or time window, planning data can be read out and taken into account which, for example, already have otherwise scheduled or blocked time windows in which, for example, treatments are being carried out or another maintenance process is running which is incompatible with the filter backwash.

Once the future time has been determined, a control instruction is generated in step S6, which should initiate the desired second operating mode, e.g. filter backwashing, at this time.

Various second operating modes can be prioritized here.

For example, both a filter backwash and a regeneration of the softener can each be determined with a necessity value of 80%. First, it can be checked whether it is possible to carry out both of these processes at the specified future time.

If this is not the case, it can be prioritized based on urgency values by selecting the second operating mode (filter backwash or softener regeneration), which is associated with a higher urgency value. In other words, the most urgent second operating mode is selected according to step S7.

In step S8, a control instruction is then issued to execute the selected second operating mode (e.g. filter backwash) at the specified future time (e.g. tomorrow at 12:30).

1. A water treatment system for producing medical solution, which is capable of being operated in a first operating mode and a second operating mode, said water treatment system comprising a control unit, wherein the control unit is programmed to detect, whether operation of the water treatment system and/or at least one component thereof in the second operating mode is required, and if operation of the water treatment system in the second operating mode has been detected to be required, to determine whether switching from the first operating mode to the second operating mode is possible at a current point in time, and if the determination is that switching to the second operating mode is possible at the current time, to issue a control instruction to the water treatment system and/or at least one component thereof to operate it in the second operating mode, and if the determination is that switching to the second operating mode is not possible at the current time, to determine a future time at which the control unit will issue a control instruction to the water treatment system and/or at least one component thereof to operate it in the second operating mode.

2. The water treatment system according to claim 1, wherein the control unit is further programmed to compare sensor data with respect to at least one system-specific and/or component-specific parameter with at least one threshold value stored in a database when determining whether operation of the water treatment system and/or at least one component thereof in the second operating mode is required.

3. The water treatment system according to claim 1, wherein the control unit is further programmed to determine, when determining whether operation of the water treatment system and/or at least one component thereof is required in the second operating mode, to determine an urgency value which reflects an urgency with which the water treatment system and/or at least one component thereof is to be operated in the second operating mode.

4. The water treatment system according to claim 3, wherein the control unit is further programmed to determine an urgency value for each of the components and/or for each of the second operating modes, taking into account sensor data regarding at least one component-specific parameter and/or at least one threshold value stored in a database, in a case in which it has been detected that it is necessary to operate a plurality of components of the water treatment system in a second operating mode, respectively.

5. The water treatment system according to claim 4, wherein the control unit is further programmed to select the component and/or the second operating mode associated with the most urgent urgency value and to issue a control instruction at the determined future time to operate the selected component in the selected second operating mode.

6. The water treatment system according to claim 1, wherein the control unit is further programmed to take into account, when determining the future time for issuing the control instruction, planning data which reflect a planned future operation of the water treatment system, wherein the planning data include at least one future time period in which the water treatment system is operated in the first operating mode or a further operating mode which prevents switching the operation of the water treatment system to the second operating mode.

7. The water treatment system according to claim 1, wherein the planning data for the operation in the second operating mode specify a certain temporal relationship to an operation in the first operating mode, in that the second operating mode is to be carried out before or after a time period in which an operation in the first operating mode takes place.

8. The water treatment system according to claim 6, wherein the control unit is further programmed to integrate a specific future time at which the control unit issues a control instruction to the water treatment system and/or at least one component thereof to operate it in the second operating mode, and thereby generate updated planning data.

9. The water treatment system according to claim 8, wherein the control unit is further programmed to determine, based on the determined future time at which the control unit issues a control instruction to the water treatment system and/or at least one component thereof to operate it in the second operation mode, at least one future time period in which another component of the water treatment system must not be operated in the second operation mode.

10. The water treatment system according to claim 1, wherein the first operating mode corresponds to an operation of the system for generating medical water for an ongoing treatment, and the second operating mode corresponds to a maintenance mode of at least one component of the water treatment system, and/or corresponds to an internal system operation for generating medical solution.

11. The water treatment system according to claim 1, wherein the control unit is configured to communicate wirelessly with the water treatment system and/or at least one sensor which provides sensor data relating to an operation of the water treatment system.

12. The water treatment system according to claim 1, wherein the control unit is completely or at least partially separate from the water treatment system and is arranged, in a smartphone, tablet or external computer.

13. A method for operating the water treatment system for producing medical solution of claim 1, said method comprising the steps:
detecting, whether operation of the water treatment system and/or at least one component thereof is required in the second operating mode, and
if operation of the water treatment system in the second operating mode has been detected to be necessary, determining whether switching from the first operating mode to the second operating mode is possible at a current time, and
if the determination is that switching to the second operating mode is possible at the current time, issuing a control instruction to the water treatment system and/or at least one component thereof to operate it in the second operating mode, and
if the determination is that switching to the second operating mode is not possible at the current time, determining a future time at which the control unit issues a control instruction to the water treatment system and/or at least one component thereof to operate it in the second operating mode.

14. A computer program product, containing machine-readable instructions which, when read by a control unit of the water treatment system of claim 1, provide the latter with the functionality of the water treatment system.

15. A data carrier containing machine-readable instructions which, when read by a control unit, a processor or a computer, enable the latter to carry out the method according to claim 13.

16. The water treatment system according to claim 1, wherein the medical solution is dialysis water.

17. The water treatment system according to claim 1, wherein the control unit is programmed to detect on the basis of sensor data whether operation of the water treatment system and/or at least one component thereof in the second operating mode is required.

18. The water treatment system according to claim 1, wherein the control unit is further programmed to determine, when determining whether operation of the water treatment system and/or at least one component thereof is required in the second operating mode taking into account sensor data with regard to at least one system-specific and/or component-specific parameter and/or at least one threshold value stored in a database for the second operating mode, to determine an urgency value which reflects an urgency with which the water treatment system and/or at least one component thereof is to be operated in the second operating mode.

19. The water treatment system according to claim 1, wherein the first operating mode corresponds to an operation of the system for generating medical water for an ongoing treatment that is a dialysis treatment, and the second operating mode corresponds to a maintenance mode of at least one component of the water treatment system that is a cleaning operation, a regeneration operation or a replacement operation and/or corresponds to an internal system operation for generating medical solution, wherein the operation is for mixing concentrate with water.

20. A method for operating a water treatment system for producing medical solution that is dialysis water, wherein the water treatment system is capable of being operated in a first operating mode and a second operating mode, said method comprising the steps:
detecting on the basis of sensor data, whether operation of the water treatment system and/or at least one component thereof is required in the second operating mode, and
if operation of the water treatment system in the second operating mode has been detected to be necessary, determining whether switching from the first operating mode to the second operating mode is possible at a current time, and
if the determination is that switching to the second operating mode is possible at the current time, issuing a control instruction to the water treatment system and/or at least one component thereof to operate it in the second operating mode, and
if the determination is that switching to the second operating mode is not possible at the current time, determining a future time at which the control unit issues a control instruction to the water treatment system and/or at least one component thereof to operate it in the second operating mode.
